(19) United States
(12) Patent Application Publication
(10) Pub. No.: US 2017/0086796 A1
(43) Pub. Date: Mar. 30, 2017

(54) ULTRASONIC RECEPTION SIGNAL CORRECTION DEVICE, ULTRASONIC MEASUREMENT APPARATUS, AND ULTRASONIC RECEPTION SIGNAL CORRECTION METHOD

(71) Applicant: SEIKO EPSON CORPORATION, Suwa-shi (JP)

(72) Inventor: Ryoki WATANABE, Suwa-shi (JP)

(73) Assignee: SEIKO EPSON CORPORATION, Tokyo (JP)

(21) Appl. No.: 15/278,710

(22) Filed: Sep. 28, 2016

(30) Foreign Application Priority Data
Sep. 29, 2015 (JP) 2015-190937

(51) Int. Cl. A61B 8/08; A61B 8/00; A61B 5/00; A61B 8/14; G01S 15/89; G01S 7/52

(52) U.S. Cl. CPC A61B 8/5269; G01S 15/8988; G01S 7/52026; A61B 5/7257; A61B 8/14; A61B 8/4444

(57) ABSTRACT

An ultrasonic measurement apparatus extracts, from a reception signal, a plurality of partial reception signals according to overlap periods that are set by shifting the time so as to overlap partially. Then, scattering attenuation correction using a scattering attenuation correction value is performed for a frequency domain signal obtained by performing a Fourier transform of each of the plurality of partial reception signals, and an inverse Fourier transform of a corrected frequency correction signal is performed. Then, partial correction signals corresponding to the respective partial reception signals are obtained by performing an inverse Fourier transform. Then, a signal obtained by combining the partial correction signals is set as a reception correction signal after correcting the reception signal. The scattering attenuation correction value is calculated from the signal strength of incident ultrasonic waves and a sum value of the signal strength of the reception signal at each time.

### BACKGROUND

1. Technical Field

The present invention relates to an ultrasonic reception signal correction device and the like for correcting a reception signal of reflected waves of ultrasonic waves from a subject.

2. Related Art

In an ultrasonic measurement apparatus that acquires biological information of a subject using ultrasonic waves, there has been a problem of acoustic shadow. Ultrasonic waves incident on the subject propagate through the subject while being reflected on the boundary surface of the body tissue, such as muscles, blood vessels, and bones. Accordingly, it is possible to know the structure of the body tissue from the reception signal of reflected waves (ultrasonic echoes) of the ultrasonic waves. However, since ultrasonic waves cannot pass through the tissue that strongly reflects the ultrasonic waves, such as a bone or a stone, reflected waves from a region behind the tissue that strongly reflects the ultrasonic waves are hardly obtained. A non-echo region or a low echo region where reflected waves are hardly obtained is called an acoustic shadow.

As a technique for reducing such an acoustic shadow, for example, there is known a method of calculating, from the average brightness of a high-brightness portion and a region behind the high-brightness portion in a tomographic image obtained from reflected waves of ultrasonic waves, an acoustic shadow effect coefficient, which is a value corresponding to the degree of presence of acoustic shadow in the region behind the high-brightness portion, and correcting the brightness of the region behind the high-brightness portion using the coefficient (refer to paragraphs [0066] to [0072] in JP-A-2005-103129).

However, the above method disclosed in JP-A-2005-103129 is to detect a low brightness region where it is thought that the acoustic shadow occurs and average brightness values including a high brightness region around the low brightness region. Accordingly, it is hard to say that the effect of reducing the acoustic shadow is sufficiently obtained.

### SUMMARY

An advantage of some aspects of the invention is to propose a new method of reducing the acoustic shadow.

A first aspect of the invention is directed to an ultrasonic reception signal correction device including: a unit that calculates an attenuation correction value using a signal strength of ultrasonic waves incident on a subject and a signal strength sum value of a reception signal obtained by receiving reflected waves from the subject; a unit that performs a Fourier transform of the reception signal; a unit that corrects a signal after the Fourier transform using the attenuation correction value; and a unit that performs an inverse Fourier transform of a signal after the correction.

As another aspect of the invention, the invention maybe configured as an ultrasonic reception signal correction method of performing arithmetic processing for correcting an ultrasonic reception signal using an arithmetic processing unit. The ultrasonic reception signal correction method includes: calculating an attenuation correction value using a signal strength of ultrasonic waves incident on a subject and a signal strength sum value of a reception signal obtained by receiving reflected waves from the subject; performing a Fourier transform of the reception signal; correcting a signal after the Fourier transform using the attenuation correction value; and performing an inverse Fourier transform of a signal after the correction.

According to the first aspect and the like of the invention, it is possible to realize a new method capable of reducing the acoustic shadow by appropriately correcting the reception signal obtained by receiving the reflected waves of ultrasonic waves. That is, the reception signal is corrected by calculating the attenuation correction value using the signal strength of the incident ultrasonic waves and the signal strength sum value of the reception signal, performing a signal obtained by performing a Fourier transform of the reception signal using the attenuation correction value, and then performing an inverse Fourier transform. In the signal obtained by performing a Fourier transform of the reception signal, a peak appears at the frequency of the reflected wave. Accordingly, by correcting the signal obtained by performing the Fourier transform, it is possible to correct the reception signal without a need to detect a region where acoustic shadow has occurred.

As a second aspect of the invention, the ultrasonic reception signal correction device according to the first aspect of the invention may be configured such that the ultrasonic reception signal correction device further includes a unit that extracts a plurality of partial reception signals from the reception signal by shifting a predetermined extraction period in a time direction, the Fourier transform, the correction, and the inverse Fourier trans form are performed for the partial reception signals, and signals after the inverse Fourier transform may be combined as a signal of the corresponding extraction period.

According to the second aspect of the invention, even in a case where a plurality of reflected waves at different depth positions are included in the reception signal, it is possible to reduce the acoustic shadow by appropriately correcting the reception signal. That is, a plurality of partial reception signals are extracted from the reception signal, and signals obtained by performing the Fourier transform, the correction, and the inverse Fourier transform for the plurality of extracted partial reception signals are combined as a signal of the corresponding extraction period, thereby correcting the reception signal. In a case where a plurality of reflected waves at different depth positions are included in the reception signal, peaks of the plurality of reflected waves appear at the same frequency in a signal obtained by performing a Fourier transform of the reception signal. Therefore, it is possible to perform correction to obtain a more appropriate reception signal by appropriately setting the extraction period so as to reduce the number of reflected waves included in the partial reception signal (ideally, so as to include only one reflected wave).

As a third aspect of the invention, the ultrasonic reception signal correction device according to the second aspect of the invention may be configured such that the calculation of the attenuation correction value is to calculate the attenuation correction value for each cumulative time at which the reception signal is received, and the correction is to correct the relevant partial reception signal using the attenuation correction value at a cumulative time corresponding to the partial reception signal.

According to the third aspect of the invention, the attenuation correction value is calculated for each cumulative time at which the reception signal is received, and the partial reception signal is corrected using the attenuation correction value at the corresponding cumulative time. Therefore, it is possible to perform appropriate correction for each partial reception signal.

As a fourth aspect of the invention, the ultrasonic reception signal correction device according to the third aspect of the invention may be configured such that a sampling unit of the cumulative time is set to be shorter than a shifting time of the extraction period, and the correction is to average the attenuation correction value at the cumulative time corresponding to the partial reception signal and correcting the partial reception signal using the average value.

According to the fourth aspect of the invention, the sampling unit of the cumulative time is shorter than the shifting time of the extraction period, and the partial reception signal is corrected using the average value of the attenuation correction value at the cumulative time corresponding to the partial reception signal.

As a fifth aspect of the invention, the ultrasonic reception signal correction device according to any one of the second to fourth aspects of the invention may be configured such that the extraction is to extract the partial reception signals by shifting the extraction period so as to overlap partially, and a signal strength of an overlapping portion between the partial reception signals is adjusted.

According to the fifth aspect of the invention, since the partial reception signals are extracted by shifting the extraction period so as to overlap partially and the signal strength of the overlapping portion between the partial reception signals is adjusted, a signal obtained by combining the signals after the inverse Fourier transform, that is, a signal obtained by correcting the reception signal, can become an appropriate signal.

A sixth aspect of the invention is directed to an ultrasonic measurement apparatus including: an ultrasonic probe that makes ultrasonic waves incident on a subject and receives reflected waves of the ultrasonic waves; and the ultrasonic reception signal correction device according to any one of the first to fifth aspects of the invention that corrects a reception signal received by the ultrasonic probe.

According to the sixth aspect of the invention, it is possible to realize the ultrasonic measurement apparatus having the effect according to any one of the first to fifth aspects of the invention.

### BRIEF DESCRIPTION OF THE DRAWINGS

The invention will be described with reference to the accompanying drawings, wherein like numbers reference like elements.

FIG. 1 is a diagram showing an example of acoustic shadow.

FIG. 2 is a simple model diagram illustrating scattering attenuation.

FIG. 3 is a simple model diagram illustrating scattering attenuation correction.

FIGS. 4A to 4D are explanatory views of examples of simulation using a scattering attenuation correction algorithm.

FIG. 5 is an explanatory view of scattering attenuation correction for a reception signal.

FIG. 6 is an explanatory view of scattering attenuation correction for a partial reception signal.

FIG. 7 is a diagram showing the functional configuration of an ultrasonic measurement apparatus.

FIG. 8 is a flowchart of the ultrasonic measurement process.

FIGS. 9A to 9D are explanatory views of experimental results.

### DESCRIPTION OF EXEMPLARY EMBODIMENTS

### Principle

An ultrasonic measurement apparatus of the present embodiment is a device that measures biological information of a subject using ultrasonic waves. By making ultrasonic waves incident on the subject from an ultrasonic probe (by transmitting ultrasonic waves to the subject from the ultrasonic probe) and performing signal processing on a reception signal of reflected waves (ultrasonic echoes), it is possible to obtain the position information of a structure in the subject or reflected wave data, such as temporal changes in the structure. Not only the reception signal itself but also images of respective modes of so-called A mode, B mode, M mode, and color Doppler mode are included in the reflected wave data. In addition, the ultrasonic measurement apparatus is also an ultrasonic reception signal correction device that corrects a reception signal of attenuated ultrasonic waves to reduce the acoustic shadow.

In an ultrasonic probe, a plurality of ultrasonic elements (ultrasonic transducers) for transmitting and receiving ultrasonic waves are arranged. Each ultrasonic element is an ultrasonic transducer for conversion between an ultrasonic wave and an electrical signal, and transmits an ultrasonic pulse signal having a frequency of several to several tens of megahertz and receives a reflected wave thereof.

### (1) Acoustic Shadow

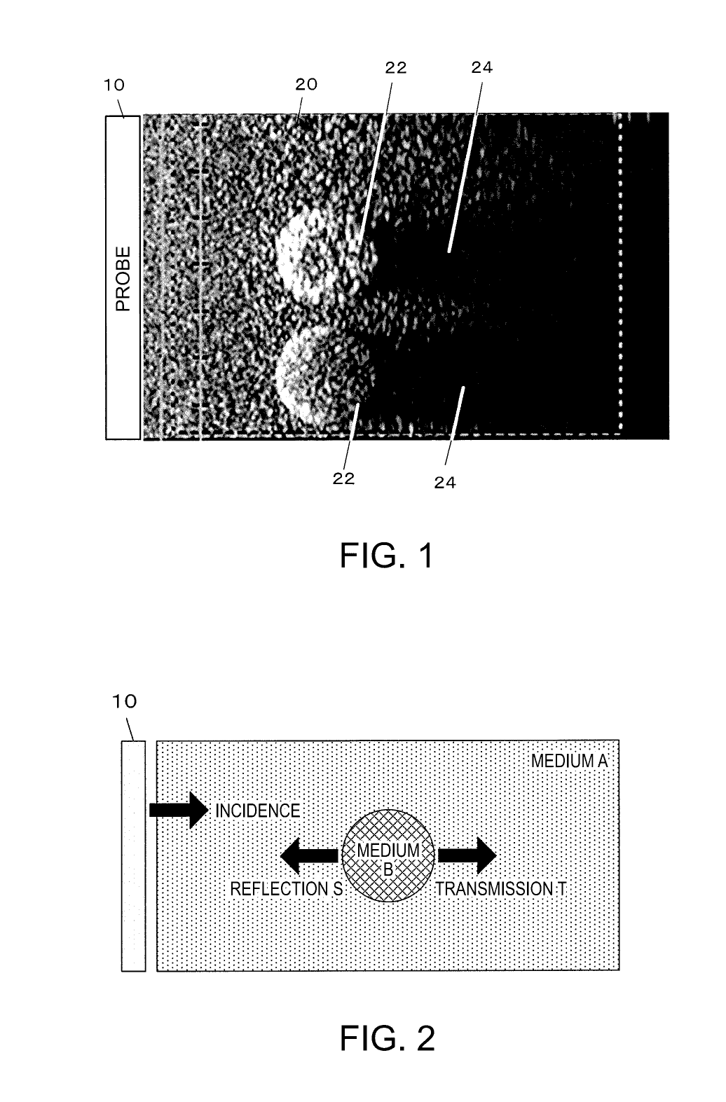

Acoustic shadow is a “strip-shaped low echo region or non-echo region occurring behind a medium that strongly reflects ultrasonic waves. FIG. 1 is an example of a B-mode image in which acoustic shadow occurs. FIG. 1 shows a B-mode image obtained by making ultrasonic waves incident on a subject 20 containing two strong reflectors 22, which strongly reflect ultrasonic waves, from an ultrasonic probe 10 in the right direction in the diagram. That is, in FIG. 1, the right direction is a direction of the depth from the surface of the subject 20, and the vertical direction is a direction along the surface of the subject 20. It can be seen that the acoustic shadow 24 having a low brightness, that is, a low reception signal strength has occurred behind the strong reflectors 22 when viewed from the ultrasonic probe 10.

(2) Causes of Acoustic Shadow

Ultrasonic waves incident on the subject propagate through the subject while being attenuated. As attenuation occurring at this time, there are mainly three types of attenuation, that is, diffusion attenuation, absorption attenuation, and scattering attenuation. The diffusion attenuation is attenuation due to sound waves spreading in a spherical shape, and the absorption attenuation is attenuation due to acoustic energy being absorbed into a medium and thermally converted. The scattering attenuation is attenuation due to a non-uniform medium. The scattering attenuation is believed to be the main cause of the acoustic shadow.

FIG. 2 is a diagram showing a simple ultrasonic wave propagation model illustrating the scattering attenuation. FIG. 2 shows a case where an ultrasonic wave from the ultrasonic probe 10 is incident on a medium A containing a medium B in the right direction in the diagram. Here, it is assumed that there is no scattering attenuation and absorption attenuation of ultrasonic waves.

The acoustic impedance Z₁ of the medium A is a product of the average density ρ₁ and the average speed of sound c₁ of the medium A, and the acoustic impedance Z₂ of the medium B is a product of the average density ρ₂ and the average speed of sound c₂ of the medium B. The acoustic impedances Z₁ and Z₂ are expressed by the following Equation (1).

Z₁=ρ₁×c₁

Z₂=ρ₂×c₂ (1)

A reflectance S when ultrasonic waves, which are incident from the ultrasonic probe 10 and propagate through the medium A, are reflected on the boundary surface of the media A and B is expressed by the following Equation (2) using the acoustic impedances Z₁ and Z₂ of the media A and B.

$$S = {( \frac{{Z}_{2} - {Z}_{1}}{{Z}_{2} + {Z}_{1}} )}^{2} \qquad ( 2 )$$

In addition, a transmittance T of ultrasonic waves passing through the boundary surface of the media A and B is expressed by the following Equation (3).

$$T = 1 - S = \frac{4 {Z}_{1} {Z}_{2}}{{( {Z}_{2} + {Z}_{1} )}^{2}} \qquad ( 3 )$$

From Equation (3), it can be seen that the reflectance S of ultrasonic waves on the boundary surface of the media A and B increases and the transmittance T of ultrasonic waves on the boundary surface of the media A and B decreases as a difference between the acoustic impedances Z₁ and Z₂ of the media A and B increases.

That is, since the signal strength of ultrasonic waves transmitted to the back side of the medium B is reduced, acoustic shadow occurs.

### (3) Algorithm for Correcting the Reception Signal Strength

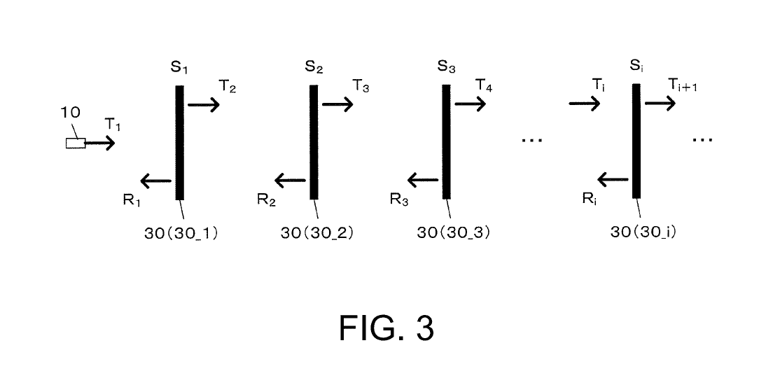

Correction of the reception signal of reflected waves of ultrasonic waves attenuated by scattering attenuation will be described. FIG. 3 is a simple ultrasonic wave propagation model illustrating the correction of the reception signal of reflected waves of ultrasonic waves. FIG. 3 shows a case where an ultrasonic wave from the ultrasonic probe 10 having a signal strength T1 is incident on a subject having a plurality of medium boundary surfaces 30 in the right direction in FIG. 3. Here, it is assumed that there is no scattering attenuation and absorption attenuation.

In the subject, a plurality of medium boundary surfaces 30_i (i=1, 2, . . . ) are present so as to face the incidence direction of ultrasonic waves, and ultrasonic waves incident from the ultrasonic probe 10 propagate while being reflected on the medium boundary surfaces 30 or being transmitted through the medium boundary surfaces 30. The reflectance Si of the i-th medium boundary surface 30_i is determined by the acoustic impedance Z of two media at the boundary as expressed by Equation (2). The signal strength (reflection intensity) Rᵢ of the reflected wave of the ultrasonic wave due to the i-th medium boundary surface 30_i is a product of the signal strength (incidence intensity) Tᵢ of the ultrasonic wave incident on a medium boundary surface i and the reflectance Sᵢ of the medium boundary surface i, and is expressed by the following Equation (4).

Rᵢ=Tᵢ×Sᵢ (4)

The incidence intensity T1 of the ultrasonic wave incident on the first medium boundary surface 30_1 is an incidence intensity T1 of the ultrasonic wave from the ultrasonic probe 10. The incidence intensity Tᵢ of the ultrasonic wave incident on the second medium boundary surface 30_i (i=2, 3, . . . ) is the transmission intensity of the previous (i−1)-th medium boundary surface 30_(i−1), and is a difference between the incidence intensity Tᵢ₋₁ of the ultrasonic wave incident on the medium boundary surface 30_(i−1) and the reflection intensity of the reflected wave from the medium boundary surface 30_(i−1). The incidence intensity Tᵢ is expressed by the following Equation (5).

Tᵢ=Tᵢ₋₁−Rᵢ₋₁ (5)

That is, the incidence intensity Tᵢ of the ultrasonic wave incident on each medium boundary surface 30_i (i=1, 2, . . . ) is expressed by the following Equation (6).

$${T}_{2} = {T}_{1} - {R}_{1} \\ {T}_{3} = {T}_{2} - {R}_{2} = ( {T}_{1} - {R}_{1} ) - {R}_{2} \\ {T}_{4} = {T}_{3} - {R}_{3} = ( {T}_{1} - {R}_{1} - {R}_{2} ) - {R}_{3} \\ ⋮ \\ {T}_{i} = {T}_{1} - {\sum }_{j = 1}^{i - 1} {T}_{j} \qquad ( 6 )$$

Then, the reflection intensity Rᵢ of the reflected wave from each medium boundary surface 30_i is the strength (reception intensity) of the reception signal in the ultrasonic probe 10. That is, the reception signal of reflected waves of ultrasonic waves for the medium boundary surface 30_i becomes an attenuated signal since the incidence intensity Ti is reduced by the reflection of some of the ultrasonic waves on the medium boundary surface 30_j (j=1, 2, . . . , i−1) up to the previous (i−1)-th medium boundary surface.

For the i-th medium boundary surface 30_i, in a case where there is no previous medium boundary surface 30_j (j=1, 2, . . . , i−1), that is, considering an ideal state in which ultrasonic waves having the incidence intensity T₁ are incident from the ultrasonic probe 10, the reflection intensity Rᵢ on the medium boundary surface 30_i is expressed by the following Equation (7).

Rᵢ=T₁×Sᵢ (7)

However, the actual reflection intensity Rᵢ of the reflected wave from the i-th medium boundary surface 30_i is smaller than the reflection intensity Rᵢ in the ideal state due to scattering attenuation, as expressed by Equation (4). Therefore, the actual reflection intensity Rᵢ is made to match the reflection intensity Rᵢ in the ideal state by multiplying the actual reflection intensity Rᵢ by a predetermined scattering attenuation correction coefficient αᵢ, as shown in the following Equation (8).

T₁×Rᵢ=αᵢ×(Tᵢ×Rᵢ) (8)

From Equation (8), the scattering attenuation correction coefficient αᵢ for the i-th medium boundary surface 30_i is expressed by the following Equation (9).

$${α}_{i} = \frac{{T}_{1}}{T - {\sum }_{J = 1}^{i - 1} {R}_{j}} \qquad ( 9 )$$

That is, a reception signal obtained by receiving the reflected wave from the i-th medium boundary surface 30_i can be corrected to become a reception signal in a case where no scattering attenuation occurs by multiplying the reception signal by the correction coefficient αᵢ.

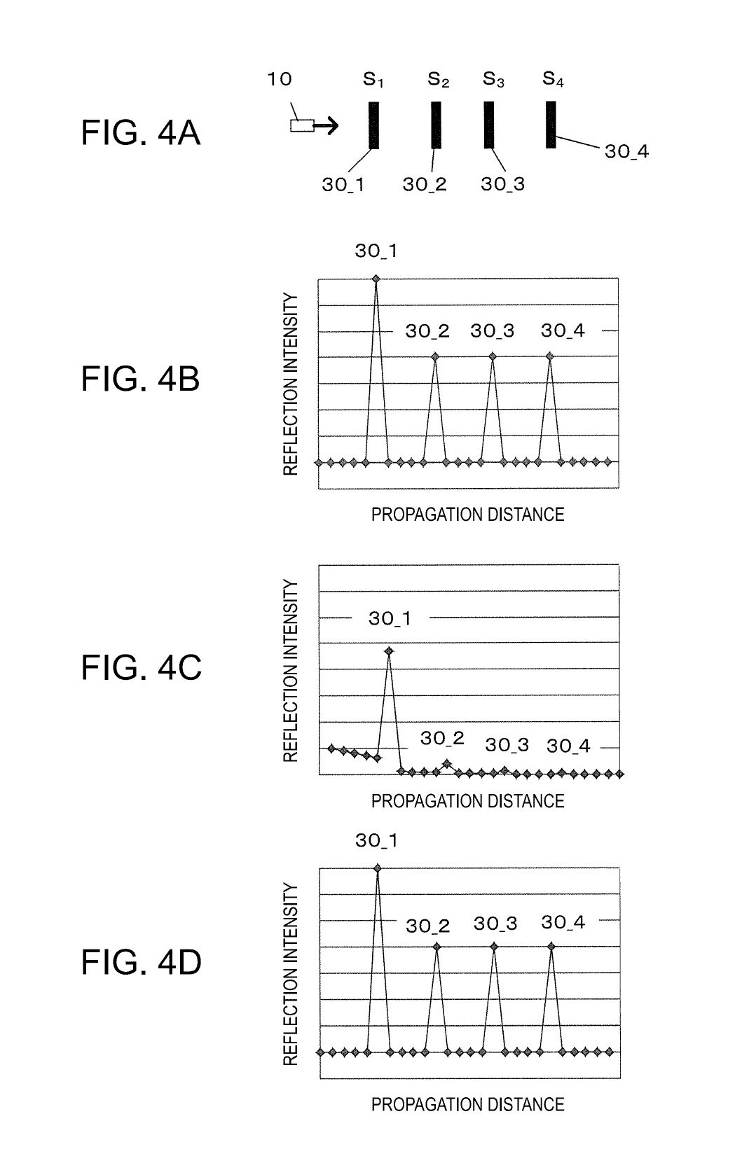

FIGS. 4A to 4D show examples of the simulation result using such a scattering attenuation correction algorithm. As a prerequisite for simulation, it is assumed that medium boundary surfaces 30_1 to 30_4 having higher reflectances S₁ to S₄ than the reflectance of a medium (virtual body of a subject), which has uniform reflectance and attenuation effect, are present in the medium. The reflectances S₁ to S₄ are set to satisfy the relationship of S₁>S₂=S₃=S₄. Then, ultrasonic waves are incident from the ultrasonic probe 10 placed on the medium boundary surface 30_1 side of the medium, reflected waves of the ultrasonic waves are received at the incidence position, and a reflection intensity corresponding to the distance from the incidence position is calculated based on the reception signal.

In FIG. 4B, a reflection intensity at a relevant position is calculated in consideration of the reflectance at only each position in the medium (in other words, by setting the reflectance or the attenuation effect at positions other than the relevant position to zero). Since the reflectance in the medium is uniform, reflection intensities at positions other than the positions of the medium boundary surfaces 30_1 to 30_4 are the same. In addition, the relationship among the positions of the medium boundary surfaces 30_1 to 30_4 is the same as S₁>S₂=S₃=S₄, which is the relationship among the reflectances S₁ to S₄. This is a reflection intensity in consideration of the reflectance at only each relevant position. Accordingly, needless to say, the reflection intensity in FIG. 4B can be said to be a “reflection intensity to be received originally” and a reflection intensity in the ideal state.

FIG. 4C shows a simulation result obtained by calculating the reflection intensity in consideration of the reflectance or the attenuation effect at all positions in the medium. This reflection intensity can be said to be a “reflection intensity of reflected waves actually received” obtained by making ultrasonic waves incident on the actual subject.

When FIG. 4C is compared with FIG. 4B, first, at a position before the position of the first medium boundary surface 30_1, it can be seen that the reflection intensity decreases with an increase in the distance since ultrasonic waves propagate through the medium having uniform reflectance and attenuation effect. Then, the reflection intensity at the position of the first medium boundary surface 30_1 is reduced (attenuated) to about half of the reflection intensity in the case shown in FIG. 4B due to the attenuation effect until the ultrasonic waves reach the position of the first medium boundary surface 30_1. In addition, as a result of the reflection of many ultrasonic wave on the medium boundary surface 30_1, the reflection intensity at a position immediately after passing through the medium boundary surface 30_1 is greatly reduced. Even after the medium boundary surface 30_1, the reflection intensity decreases with an increase in the distance since the ultrasonic waves propagate through the medium having uniform reflectance and attenuation effect. For this reason, the reflection intensity at the position of the second medium boundary surface 30_2 appears slightly, the magnitude is greatly reduced (attenuated) compared with that in the case shown in FIG. 4B. Compared with the medium, ultrasonic waves are greatly reflected on the medium boundary surface 30_2. Accordingly, the reflection intensity at subsequent positions are further reduced.

FIG. 4D is a signal strength (reflection intensity) when performing scattering attenuation correction of the present embodiment for the reflection intensity of the simulation result shown in FIG. 4C. That is, for each of the positions of the four medium boundary surfaces 30_1 to 30_4, the scattering attenuation correction value αᵢ is calculated from Equation (9) using the reflection intensity Rᵢ, the reflection intensity Rᵢ₋₁ on each medium boundary surface 30 on the front side (ultrasonic probe 10 side), and the incidence intensity T₁ of the ultrasonic wave. Then, a signal strength after correction is calculated by multiplying the reflection intensity Rᵢ by the scattering attenuation correction value αᵢ.

When FIG. 4D is compared with FIG. 4C, the reflection intensity is corrected so as to increase all of the positions of the four medium boundary surfaces 30_1 to 30_4. When FIG. 4D is compared with FIG. 4B, the reflection intensities at all of the positions of the four medium boundary surfaces 30_1 to 30_4 in FIG. 4D are almost the same as those in FIG. 4B. That is, correction approaching the “ideal reflection intensity” is realized.

From the simulation result, it can be said that the scattering attenuation correction of the present embodiment can be applied to the correction of scattering attenuation or absorption attenuation. This is because the incidence intensity of the ultrasonic wave incident on the certain medium boundary surface 30_i is the transmission intensity of the medium boundary surface 30_(i−1) before the medium boundary surface 30_i (on the ultrasonic probe 10 side) in Equation (9) of the scattering attenuation correction value αᵢ. That is, this is because the transmission intensity Tᵢ and the reflection intensity Rᵢ on the medium boundary surface 30_i appear as values including attenuation due to a medium between the medium boundary surface 30_i and the medium boundary surface 30_(i−1) before the medium boundary surface 30_i (ultrasonic probe 10 side) (refer to FIG. 3).

### (4) Application to an Ultrasonic Measurement Apparatus

An ultrasonic measurement apparatus makes ultrasonic pulses incident on a subject. However, since a variety tissues are included in the body as a subject, the reflection position of ultrasonic waves in the subject, that is, the position of a medium boundary surface, is not known. Accordingly, the above-described scattering attenuation correction is performed for a signal in the frequency domain obtained by performing a Fourier transform of a reception signal that is a signal in the time domain, and a signal after the correction is returned to the signal in the time domain by performing an inverse Fourier transform. Within the body that is a subject, a number of medium boundary surfaces due to body tissues are present, and the positions thereof are not known. Accordingly, since a reception signal becomes a composite signal of reflected waves from the plurality of medium boundary surfaces, it is very difficult to determine from which medium boundary surface (that is, depth position) an ultrasonic wave has been reflected. For this reason, the reception signal is extracted so as to be divided into a plurality of partial reception signals, scattering attenuation correction is performed for each of the partial reception signals, and then signals after correction are combined.

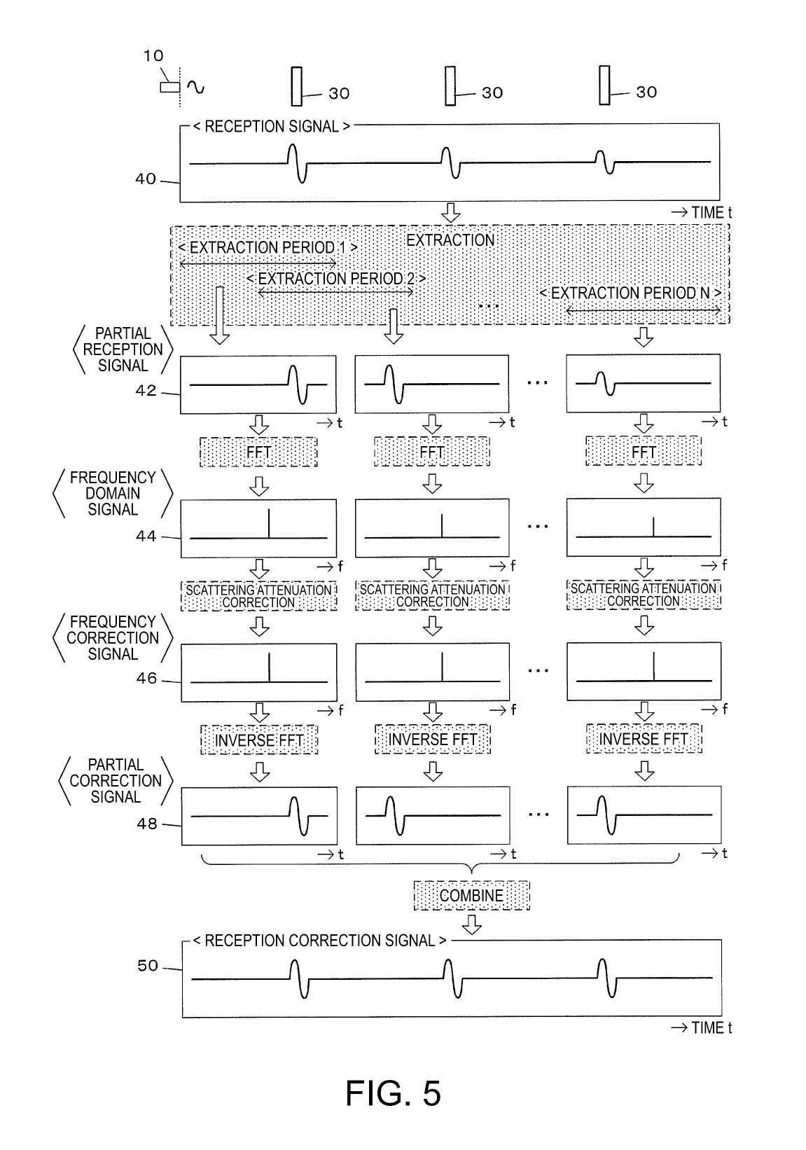

FIG. 5 is a schematic diagram of the procedure of scattering attenuation correction for a reception signal in the ultrasonic measurement apparatus. The procedure is in the order from top to bottom. First, ultrasonic pulses are incident on the subject 20 from the ultrasonic probe 10. A number of medium boundary surfaces 30 are present in the subject 20. In FIG. 5, only the three medium boundary surfaces 30 are shown. In practice, however, four or more medium boundary surfaces 30 may be present or two or less medium boundary surfaces 30 may be present, and the positions thereof are not known.

In response to the incidence of the ultrasonic pulses, a reception signal 40 is obtained in the ultrasonic probe 10. In the reception signal 40, a reflected wave from the relevant medium boundary surface 30 is generated at a time t corresponding to the depth position of the medium boundary surface 30. In general, the signal strength of the reflected wave included in the reception signal 40 decreases due to attenuation as the depth position deepens.

From the reception signal 40, partial reception signals 42 are extracted during a plurality of set extraction periods. It is assumed that the extraction periods are the same length and the length is larger than the period of the ultrasonic pulse. The extraction periods are set so as to be shifted from each other in the time direction so that parts of the extraction periods overlap each other. Accordingly, needless to say, parts of the partial reception signals 42 in the extraction periods partially overlapping each other also overlap each other.

Then, a frequency domain signal 44 that is a signal in the frequency domain is generated by performing a Fourier transform (“FFT” in the diagram) for the extracted partial reception signals 42. In a case where a reflected wave of the ultrasonic pulse is included in each partial reception signal 42, a large peak appears at a frequency f₀ of the ultrasonic pulse in the frequency domain signal 44 obtained by performing a Fourier transform of the reflected wave.

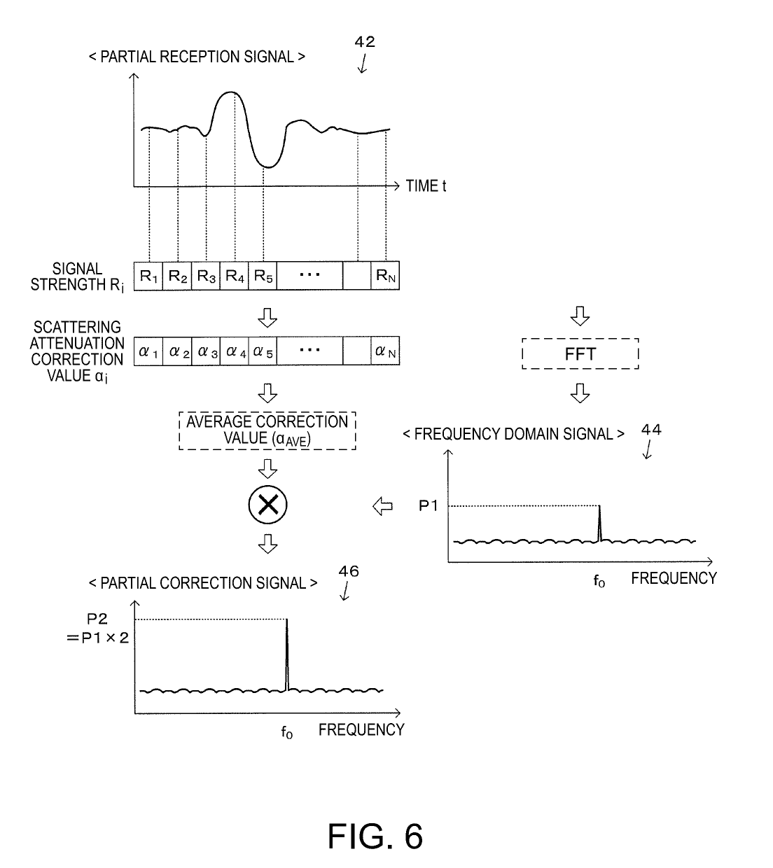

Then, a frequency correction signal 46 is generated by performing scattering attenuation correction for the frequency domain signal 44. FIG. 6 is an explanatory view of detailed scattering attenuation correction for the partial reception signal. Discrete value data of the signal strength Rᵢ (i=1, 2, . . . ) is generated by performing sampling at predetermined sampling intervals (sampling unit) Ts for the partial reception signal 42. As the signal strength Rᵢ, signal strengths R₁, R₂, . . . , R_{N} are set in order of shallow depth position (early reception time). By regarding the signal strength Rᵢ, which is the discrete value, as the reflection intensity Rᵢ on the medium boundary surface, the scattering attenuation correction value αᵢ corresponding to each signal strength Rᵢ is calculated using Equation (9). Then, the average correction value α_{AVE} that is an average value of the scattering attenuation correction value αᵢ is calculated. Then, the frequency correction signal 46 is generated by multiplying the frequency domain signal 44 by the average correction value α_{AVE}. The frequency correction signal 46 is a signal obtained by multiplying the peak magnitude in the frequency domain signal 44 by the average correction value α_{AVE}.

Then, referring back to FIG. 5, a partial correction signal 48 that is a signal in the time domain is generated by performing an inverse Fourier transform (“inverse FFT” in the diagram) of the frequency correction signal 46. Then, by combining the respective partial correction signals 48, a reception correction signal 50 that is a signal obtained by performing scattering attenuation correction for the reception signal 40 is finally generated.

### Functional Configuration

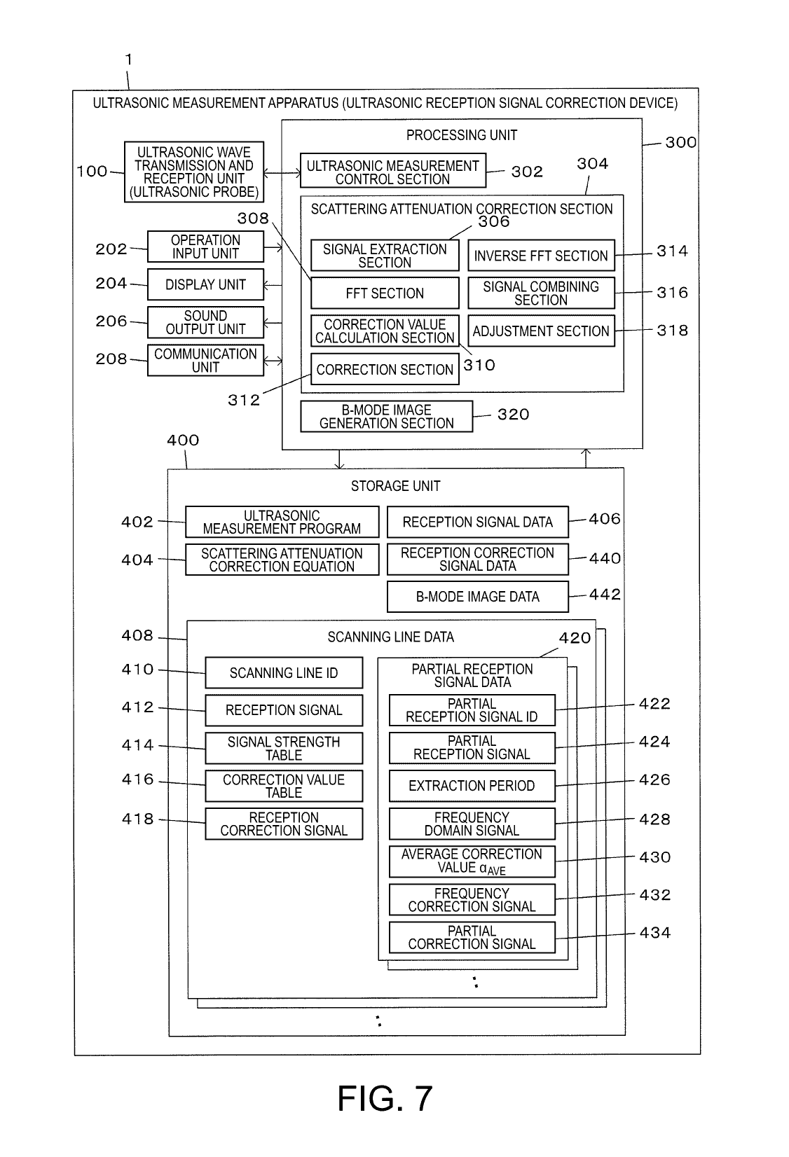

FIG. 7 is a diagram showing the functional configuration of an ultrasonic measurement apparatus 1. As shown in FIG. 7, the ultrasonic measurement apparatus 1 includes an ultrasonic wave transmission and reception unit 100, an operation input unit 202, a display unit 204, a sound output unit 206, a communication unit 208, a processing unit 300, and a storage unit 400. Since the ultrasonic measurement apparatus 1 has a function of an ultrasonic reception signal correction device, the ultrasonic measurement apparatus 1 is also an ultrasonic reception signal correction device.

The ultrasonic wave transmission and reception unit 100 is an ultrasonic probe, and has a plurality of ultrasonic elements for transmitting and receiving ultrasonic waves. Each ultrasonic element transmits an ultrasonic pulse according to a pulse voltage input from the processing unit 300, receives reflected waves of ultrasonic waves, converts the reflected waves into a reflected wave signal that is an electrical signal, and outputs the reflected wave signal to the processing unit 300.

The operation input unit 202 is implemented by input devices, such as button switches, a touch panel, and various sensors, and outputs an operation signal corresponding to an operation to the processing unit 300. The display unit 204 is implemented by a display device, such as a liquid crystal display (LCD), and performs various kinds of display based on the display signal from the processing unit 300. The sound output unit 206 is implemented by a sound output device, such as a speaker, and outputs various sounds based on the sound signal from the processing unit 300. The communication unit 208 is implemented by a wireless communication device, such as a wireless local area network (LAN) or Bluetooth (registered trademark), or a wired communication device, such as a modem, a wired communication cable jack, or a control circuit, and performs communication with an external device by being connected to a predetermined communication circuit.

The processing unit 300 is implemented by a microprocessor, such as a central processing unit (CPU) or a graphics processing unit (GPU) , or an electronic component, such as an application specific integrated circuit (ASIC), a field programmable gate array (FPGA), or an integrated circuit (IC) memory. The processing unit 300 controls the operation of the ultrasonic measurement apparatus 1 by executing various kinds of arithmetic processing based on a program or data stored in the storage unit 400, an operation signal from the operation input unit 202, or the like. The processing unit 300 includes an ultrasonic measurement control section 302, a scattering attenuation correction section 304, and a B-mode image generation section 320.

The ultrasonic measurement control section 302 controls the transmission and reception of ultrasonic waves by the ultrasonic wave transmission and reception unit 100. That is, a pulse voltage for giving an instruction on a transmission timing for each ultrasonic element is generated, and is output to the ultrasonic wave transmission and reception unit 100. By performing amplification processing or filtering processing, A/D conversion processing, and reception focusing processing on the reflected wave signal of ultrasonic waves input from the ultrasonic wave transmission and reception unit 100, reception signal data 406 is generated. The reception signal data 406 is data of a reception signal for each scanning line.

The scattering attenuation correction section 304 includes a signal extraction section 306, an FFT section 308, a correction value calculation section 310, a correction section 312, an inverse FFT section 314, a signal combining section 316, and an adjustment section 318, and generates a reception correction signal by performing scattering attenuation correction for the reception signal for each scanning line (refer to FIGS. 5 and 6). The generated reception correction signal for each scanning line is stored as reception correction signal data 440.

For the reception signal, the signal extraction section 306 extracts a plurality of partial reception signals partially overlapping each other while shifting the extraction period in a time direction.

The FFT section 308 generates a frequency domain signal by performing a Fourier transform of the partial reception signal.

The correction value calculation section 310 calculates the scattering attenuation correction value αᵢ for the frequency domain signal. That is, data of a discrete value of the signal strength Rᵢ (i=1, 2, . . . ) is generated by performing sampling at predetermined sampling intervals for the reception signal. Then, by regarding the signal strength Rᵢ as the reflection intensity Rᵢ on the medium boundary surface, the scattering attenuation correction value αᵢ corresponding to each signal strength Rᵢ is calculated using Equation (9). Equation (9) is stored as a scattering attenuation correction equation 404. Then, for each partial reception signal, an average correction value α_{AVE} that is an average value of the scattering attenuation correction value αᵢ corresponding to an extraction period is calculated.

For each partial reception signal, the correction section 312 generates a frequency correction signal by correcting the frequency domain signal by multiplying the frequency domain signal by the average correction value α_{AVE}.

The inverse FFT section 314 generates a partial correction signal by performing an inverse Fourier transform of the frequency correction signal.

The signal combining section 316 generates a reception correction signal by combining partial correction signals corresponding to the respective reception signals.

The adjustment section 318 adjusts the signal strength of the overlapping portion between the partial reception signals so as to weaken, for example. Specifically, the adjustment section 318 performs adjustment for the frequency domain signal, and the FFT section 308 performs a Fourier transform of the adjusted frequency domain signal. In addition, the adjustment section 318 performs adjustment for the reception correction signal, and the signal combining section 316 combines the respective adjusted reception correction signals.

The B-mode image generation section 320 generates a B-mode image based on the reception signal (reception correction signal) corrected by the scattering attenuation correction section 304. The generated B-mode image is stored as B-mode image data 442.

The storage unit 400 is implemented by a storage device, such as a read only memory (ROM), a random access memory (RAM), or a hard disk. The storage unit 400 stores a system program required for the processing unit 300 to perform overall control of the ultrasonic measurement apparatus 1, other programs, data, and the like, and is used as a working area of the processing unit 300. In addition, results of calculation performed by the processing unit 300, operation data from the operation input unit 202, and the like are temporarily stored in the storage unit 400. An ultrasonic measurement program 402, the scattering attenuation correction equation 404, the reception signal data 406, scanning line data 408, the reception correction signal data 440, the B-mode image data 442 are stored in the storage unit 400.

The scanning line data 408 is data for each scanning line that is generated in the process of scattering attenuation correction performed by the scattering attenuation correction section 304. A reception signal 412, a signal strength table 414 that is discrete value data of the signal strength Rᵢ obtained by sampling the reception signal 412 at predetermined sampling intervals, a correction value table 416 that is data of the scattering attenuation correction value αᵢ corresponding to each signal strength Rᵢ of the signal strength table 414, partial reception signal data 420, and a reception correction signal 418 obtained by performing scattering attenuation correction for the reception signal 412 are stored so as to match a scanning line ID 410 that is an identification number of the relevant scanning line.

The partial reception signal data 420 is data for each partial reception signal extracted from the reception signal. A partial reception signal 424, an extraction period 426, a frequency domain signal 428 obtained by performing a Fourier transform of the partial reception signal 424, an average correction value 430 that is an average value of the scattering attenuation correction value αᵢ of each signal strength Rᵢ in the extraction period 426, a frequency correction signal 432 obtained by correcting the frequency domain signal 428 using the average correction value 430, and a partial correction signal 434 obtained by performing an inverse Fourier transform of the frequency correction signal 432 are stored so as to match a partial reception signal ID 422 that is an identification number of the relevant partial reception signal.

### Process Flow

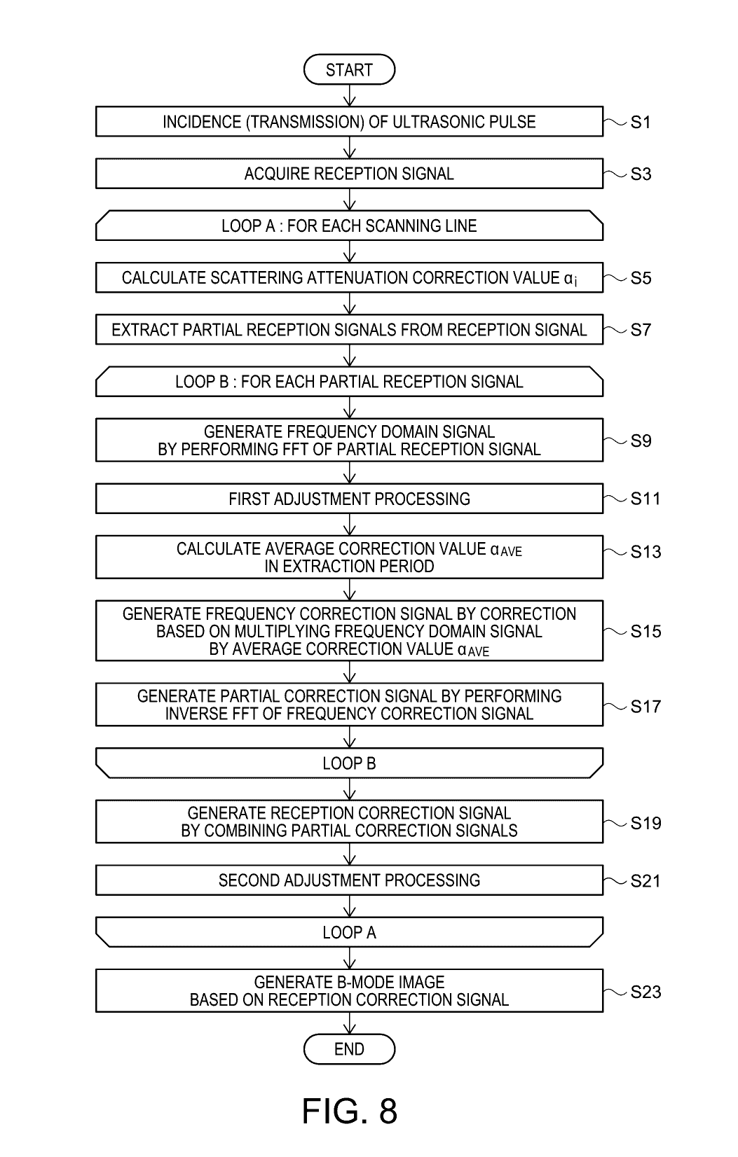

FIG. 8 is a flowchart illustrating the ultrasonic measurement process. This process is a process realized when the processing unit 300 executes the ultrasonic measurement program 402.

First, the ultrasonic measurement control section 302 controls the ultrasonic wave transmission and reception unit 100 to transmit an ultrasonic pulse to a subject (step S1). Then, reflected waves thereof are received, thereby acquiring a reception signal (reception signal data 406) (step S3). At this time, the reception signal 412 for each scanning line can be acquired by scanning the surface position of the subject.

Then, processing of a loop A for each scanning line is performed. In the loop A, the correction value calculation section 310 generates discrete value data of the signal strength Rᵢ by performing sampling at predetermined sampling intervals for the reception signal 412 of the target scanning line. Then, the scattering attenuation correction value αᵢ corresponding to each signal strength Rᵢ is calculated (step S5). Then, the signal extraction section 306 sets the extraction periods 426 such that parts of the extraction periods 426 overlap each other while shifting a predetermined time in the time direction, and extracts the partial reception signal 424 corresponding to each extraction period 426 from the reception signal 412 (step S7).

Then, processing of a loop B for each partial reception signal 424 is performed. In the loop B, the FFT section 308 generates the frequency domain signal 428 by performing a Fourier transform of the target partial reception signal 424 (step S9). Then, the adjustment section 318 performs first adjustment processing, which is for adjusting the signal strength, for the frequency domain signal 428 (step S11). The first adjustment processing is for preventing a signal value from being saturated in subsequent arithmetic processing (preventing the signal value from reaching the upper limit or the lower limit of calculation), for example, by multiplying the signal value by a predetermined value uniformly. Then, the correction value calculation section 310 calculates the average correction value α_{AVE} 430 that is an average value of the scattering attenuation correction value αᵢ corresponding to the extraction period 426 of the target partial reception signal 424 (step S13). Then, the correction section 312 performs correction by multiplying the frequency domain signal 428 by the average correction value α_{AVE} 430, thereby generating the frequency correction signal 432 (step S15). Then, the inverse FFT section 314 generates the partial correction signal 434 by performing an inverse Fourier transform of the frequency correction signal 432 (step S17). In this manner, the loop B is performed.

After performing the processing of the loop B for all partial reception signals 424, the signal combining section 316 generates a reception correction signal (reception correction signal data 440) by combining the partial correction signals 434 (step S19). Then, the adjustment section 318 performs second adjustment processing, which is for adjusting the signal strength, for the reception correction signal (step S21). The second adjustment processing is for preventing a signal value from being saturated in subsequent arithmetic processing (preventing the signal value from reaching the upper limit or the lower limit of calculation), for example, by multiplying the signal value by a predetermined value uniformly. In this manner, the loop A is performed.

After performing the processing of the loop A for all scanning lines, the B-mode image generation section 320 generate a B-mode image (B-mode image data 442) based on the reception correction signal for each scanning line (reception correction signal data 440) (step S23).

### Experimental Results

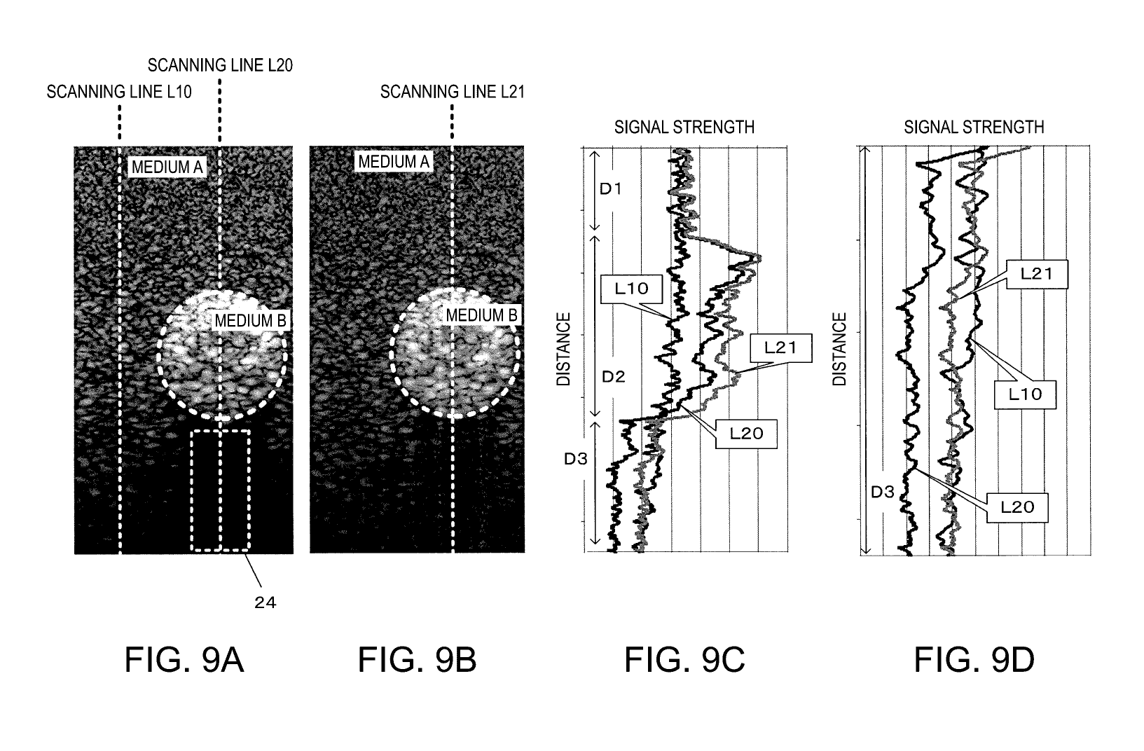

FIGS. 9A to 9D are diagrams showing examples of the processing result of the ultrasonic measurement apparatus 1. FIG. 9A is a B-mode image obtained from a reception signal of reflected waves of ultrasonic waves incident on the medium A configured to include the medium B that is a strong reflector. Here, scattering attenuation correction is not performed. FIG. 9B is a B-mode image obtained from a signal that is obtained by performing scattering attenuation correction for the reception signal obtained in FIG. 9A using the ultrasonic measurement apparatus 1. When FIGS. 9A and 9B are compared, an acoustic shadow 24 occurs on the back side of the medium B in FIG. 9A. However, as shown in FIG. 9B, it can be seen that the acoustic shadow is reduced by performing the scattering attenuation correction of the present embodiment.

FIG. 9C is a “graph of the signal strength with respect to a depth position” along scanning lines L10, L20, and L21 in FIGS. 9A and 9B. Specifically, three graphs of a graph of the average signal strength of 20 scanning lines including the scanning line L10, which does not pass through the medium B, at the center and a graph of the average signal strength of 20 scanning lines including the scanning line L20, which passes through the medium B, at the center (these graphs are shown in FIG. 9A) and a graph of the signal strength of the scanning line L21, that is, a graph of the signal strength obtained by performing scattering attenuation correction for the signal strength of the scanning line L20 (this graph is shown in FIG. 9B) are shown so as to overlap each other. FIG. 9D is graphs obtained by enlarging a range, which corresponds to the “acoustic shadow 24” in FIG. 9A, in the graphs shown in FIG. 9C.

According to FIGS. 9C and 9D, the signal strength of the scanning line L10 decreases (attenuates) gradually as the depth position becomes deep, and the degree of attenuation is uniform. This is believed to be due to diffusion attenuation and absorption attenuation.

When the signal strengths of the scanning lines L10 and L20 are compared, it can be seen that the signal strengths of the scanning lines L10 and L20 are approximately the same in a depth range D1 before reaching the medium B but the signal strength of the scanning line L20 increases abruptly at the boundary position between the media A and B and ultrasonic waves are strongly reflected. Then, the signal strength of the scanning line L20 decreases (attenuates) gradually in a depth range D2 corresponding to the inside of the medium B, and becomes lower (smaller) than the signal strength of the scanning line L10 on the whole in a depth range D3 corresponding to the back side of the medium B. That is, the acoustic shadow 24 occurs on the back side of the medium B.

In addition, the signal strengths of the scanning lines L20 and L21 are approximately the same in the depth range D1 before reaching the medium B and the depth range D2 corresponding to the inside of the medium B. However, in the depth range D3 corresponding to the back side of the medium B, the signal strength of the scanning line L21 is higher (larger) than the signal strength of the scanning line L20, and is approximately the same as the signal strength of the scanning line L10.

That is, as can be seen through the comparison between FIGS. 9A and 9B, it can be seen that the acoustic shadow 24 is reduced by the scattering attenuation correction of the present embodiment.

### Effects

Thus, according to the ultrasonic measurement apparatus 1 of the present embodiment, it is possible to reduce the acoustic shadow by appropriately correcting a reception signal obtained by receiving the reflected waves of ultrasonic waves.

That is, a plurality of partial reception signals according to overlapping periods, which are set by shifting the time so as to overlap partially, are extracted from the reception signal obtained by receiving reflected waves of ultrasonic waves incident on the subject. Then, scattering attenuation correction using the scattering attenuation correction value α is performed for a frequency domain signal obtained by performing a Fourier transform of each of the plurality of partial reception signals, and an inverse Fourier transform of the corrected frequency correction signal is performed. Then, a signal obtained by combining the partial correction signals after the inverse Fourier transform corresponding to the respective partial reception signals is set as a reception correction signal obtained by correcting each reception signal. The scattering attenuation correction value α is calculated from the signal strength of incident ultrasonic waves and a sum value of the signal strength of the reception signal at each time.

A subject includes a number of medium boundary surfaces 30. For this reason, reflected waves at a plurality of different depth positions are included in a reception signal, and the depth position is unknown. In a signal obtained by performing a Fourier transform of the reception signal, a peak appears at the frequency of the reflected wave. Accordingly, by correcting the signal obtained by performing the Fourier transform, it is possible to correct the reception signal without detecting a region where acoustic shadow has occurred. In addition, by correcting partial reception signals with a small number of reflected waves and combining the partial reception signals, it is possible to correct the reception signal more appropriately.

In addition, it should be understood that embodiments to which the invention can be applied are not limited to the embodiment described above and various modifications can be made without departing from the spirit and scope of the invention.

The entire disclosure of Japanese Patent Application No. 2015-190937 filed on Sep. 29, 2015 is expressly incorporated by reference herein.

What is claimed is:

1. An ultrasonic reception signal correction device, comprising:
a processing unit configured to calculate an attenuation correction value using a signal strength of ultrasonic waves incident on a subject and a signal strength sum value of a reception signal obtained by receiving reflected waves from the subject,
perform a Fourier transform of the reception signal, correct a signal after the Fourier transform using the attenuation correction value, and
perform an inverse Fourier transform of a signal after the correction.

2. The ultrasonic reception signal correction device according to claim 1, further comprising:
the processor configured to extract a plurality of partial reception signals from the reception signal by shifting a predetermined extraction period in a time direction,
wherein the Fourier transform, the correction, and the inverse Fourier transform are performed for the partial reception signals, and
signals after the inverse Fourier transform are combined as a signal of the corresponding extraction period.

3. The ultrasonic reception signal correction device according to claim 2,
wherein the calculation of the attenuation correction value is to calculate the attenuation correction value for each cumulative time at which the reception signal is received, and
the correction is to correct the relevant partial reception signal using the attenuation correction value at a cumulative time corresponding to the partial reception signal.

4. The ultrasonic reception signal correction device according to claim 3,
wherein a sampling unit of the cumulative time is set to be shorter than a shifting time of the extraction period, and
the correction is to average the attenuation correction value at the cumulative time corresponding to the partial reception signal and correct the partial reception signal using the average value.

5. The ultrasonic reception signal correction device according to claim 2,
wherein the extraction is to extract the partial reception signals by shifting the extraction period so as to overlap partially, and
a signal strength of an overlapping portion between the partial reception signals is adjusted.

6. An ultrasonic measurement apparatus, comprising:
an ultrasonic probe that makes ultrasonic waves incident on a subject and receives reflected waves of the ultrasonic waves; and
the ultrasonic reception signal correction device according to claim 1 that corrects a reception signal received by the ultrasonic probe.

7. An ultrasonic measurement apparatus, comprising:
an ultrasonic probe that makes ultrasonic waves incident on a subject and receives reflected waves of the ultrasonic waves; and
the ultrasonic reception signal correction device according to claim 2 that corrects a reception signal received by the ultrasonic probe.

8. An ultrasonic measurement apparatus, comprising:
an ultrasonic probe that makes ultrasonic waves incident on a subject and receives reflected waves of the ultrasonic waves; and
the ultrasonic reception signal correction device according to claim 3 that corrects a reception signal received by the ultrasonic probe.

9. An ultrasonic measurement apparatus, comprising:
an ultrasonic probe that makes ultrasonic waves incident on a subject and receives reflected waves of the ultrasonic waves; and
the ultrasonic reception signal correction device according to claim 4 that corrects a reception signal received by the ultrasonic probe.

10. An ultrasonic measurement apparatus, comprising:
an ultrasonic probe that makes ultrasonic waves incident on a subject and receives reflected waves of the ultrasonic waves; and
the ultrasonic reception signal correction device according to claim 5 that corrects a reception signal received by the ultrasonic probe.

11. An ultrasonic reception signal correction method of performing arithmetic processing for correcting an ultrasonic reception signal using an arithmetic processing unit, the method comprising:
calculating an attenuation correction value using a signal strength of ultrasonic waves incident on a subject and a signal strength sum value of a reception signal obtained by receiving reflected waves from the subject;
performing a Fourier transform of the reception signal;
correcting a signal after the Fourier transform using the attenuation correction value; and
performing an inverse Fourier transform of a signal after the correction.
